SEQUENCE LISTING (30 sequences)

SEQ ID NO: 1 - protein, 15 aa, Artificial Sequence
  Description of Artificial Sequence; note = synthetic construct
SAYAATVRGPLSSAS

SEQ ID NO: 2 - protein, 6 aa, Artificial Sequence
  Description of Artificial Sequence; note = synthetic construct
YAATDR

SEQ ID NO: 3 - protein, 15 aa, Artificial Sequence
  Description of Artificial Sequence; note = synthetic construct
SSQVVGVPQLMQSSP

SEQ ID NO: 4 - protein, 15 aa, Artificial Sequence
  Description of Artificial Sequence; note = synthetic construct
DRVPLVHVIFNSFGY

SEQ ID NO: 5 - protein, 15 aa, Artificial Sequence
  Description of Artificial Sequence; note = synthetic construct
RNQGPVKMVFPIAPS

SEQ ID NO: 6 - protein, 12 aa, Artificial Sequence
  Description of Artificial Sequence; note = synthetic construct
EGQFTFPRGASE

SEQ ID NO: 7 - protein, 15 aa, Artificial Sequence
  Description of Artificial Sequence; note = synthetic construct
LAVRRYALGNGYDWS

SEQ ID NO: 8 - protein, 7 aa, Artificial Sequence
  Description of Artificial Sequence; note = synthetic construct
LAARKYA

SEQ ID NO: 9 - protein, 7 aa, Artificial Sequence
  Description of Artificial Sequence; note = synthetic construct
QSRLTAR

SEQ ID NO: 10 - protein, 5 aa, Artificial Sequence
  Description of Artificial Sequence; note = synthetic construct
QGYDS

SEQ ID NO: 11 - protein, 7 aa, Artificial Sequence
  Description of Artificial Sequence; note = synthetic construct
LAARHYA

SEQ ID NO: 12 - protein, 5 aa, Artificial Sequence
  Description of Artificial Sequence; note = synthetic construct
AVRRY

SEQ ID NO: 13 - protein, 9 aa, Artificial Sequence
  Description of Artificial Sequence; note = synthetic construct
QARLAARPY

SEQ ID NO: 14 - protein, 9 aa, Artificial Sequence
  Description of Artificial Sequence; note = synthetic construct
VPQIEVTFD

SEQ ID NO: 15 - protein, 5 aa, Artificial Sequence
  Description of Artificial Sequence; note = synthetic construct
VHEIF

SEQ ID NO: 16 - protein, 4 aa, Artificial Sequence
  Description of Artificial Sequence; note = synthetic construct
VIFN

SEQ ID NO: 17 - protein, 4 aa, Artificial Sequence
  Description of Artificial Sequence; note = synthetic construct
LVHI

SEQ ID NO: 18 - protein, 5 aa, Artificial Sequence
  Description of Artificial Sequence; note = synthetic construct
HVNSF

SEQ ID NO: 19 - protein, 7 aa, Artificial Sequence
  Description of Artificial Sequence; note = synthetic construct
HVAVNSF

SEQ ID NO: 20 - protein, 15 aa, Artificial Sequence
  Description of Artificial Sequence; note = synthetic construct
AVLVGSLGGFSRAIP

SEQ ID NO: 21 - protein, 15 aa, Artificial Sequence
  Description of Artificial Sequence; note = synthetic construct
AAVVPAHEVRFVPGS

SEQ ID NO: 22 - protein, 15 aa, Artificial Sequence
  Description of Artificial Sequence; note = synthetic construct
SWSLGLGWPGHTSRG

SEQ ID NO: 23 - protein, 15 aa, Artificial Sequence
  Description of Artificial Sequence; note = synthetic construct
PFARAPVEHHDVVGL

SEQ ID NO: 24 - protein, 15 aa, Artificial Sequence
  Description of Artificial Sequence; note = synthetic construct
FVLVRDTFPSSVCCP

SEQ ID NO: 25 - protein, 15 aa, Artificial Sequence
  Description of Artificial Sequence; note = synthetic construct
RVPPRYHAKISPMVK

SEQ ID NO: 26 - protein, 15 aa, Artificial Sequence
  Description of Artificial Sequence; note = synthetic construct
GQRSSLAVVRYSSGA

SEQ ID NO: 27 - protein, 15 aa, Artificial Sequence
  Description of Artificial Sequence; note = synthetic construct
AEPSWVVSRYHRSYV

SEQ ID NO: 28 - protein, 15 aa, Artificial Sequence
  Description of Artificial Sequence; note = synthetic construct
RTWDSALVPANHVFV

SEQ ID NO: 29 - protein, 5 aa, Artificial Sequence
  Description of Artificial Sequence; note = synthetic construct
CCAAT

SEQ ID NO: 30 - protein, 7 aa, Artificial Sequence
  Description of Artificial Sequence; note = synthetic construct
FTFPRGA

(19) United States
(12) Patent Application Publication
(10) Pub. No.: US 2010/0137221 A1
(43) Pub. Date: Jun. 3, 2010

(54) PEPTIDES THAT INTERACT WITH TOPOISOMERASE I AND METHODS THEREOF

(75) Inventors: Louis R. Barrows, Salt Lake City, UT (US); Christopher D. Pond, Salt Lake City, UT (US)

Correspondence Address: Ballard Spahr LLP, SUITE 1000, 999 PEACHTREE STREET, ATLANTA, GA, 30309-3915, US

(73) Assignee: UNIVERSITY UTAH RESEARCH FOUNDATION, Salt Lake City, UT (US)

(21) Appl. No.: 12/525,216

(22) PCT Filed: Feb. 27, 2008
(86) PCT No.: PCT/US08/55089

§ 371 Date: Feb. 8, 2010

Related U.S. Application Data
(60) Provisional application No. 60/891,823, filed on Feb. 27, 2007.

(51) Int. Cl. A61K 38/10; C07K 7/08; C07K 7/06; C07K 5/10; C12N 15/63; C12N 5/00; A61K 38/08; A61P 35/00; A61K 38/07

(52) U.S. Cl. 514/14; 530/326; 530/329; 530/327; 530/330; 530/328; 435/320.1; 435/325; 514/17; 514/16; 514/15; 514/18

(57) ABSTRACT

Disclosed are compositions and methods for treating cancer comprising peptides that can act synergistically with chemotherapeutic agents.

### I. PRIORITY STATEMENT

This application claims priority to U.S. Provisional Patent Application No. 60/891,823, which was filed on Feb. 27, 2007, herein incorporated by reference in its entirety.

### II. BACKGROUND

The camptothecin (CPT) class of anticancer drugs has recently become important in the treatment of several types of cancers. Topotecan and irinotecan, two analogs of CPT, are in clinical use. In recurrent ovarian cancer, topotecan possesses anti-tumor activity similar to paclitaxel, with non-overlapping side effects and is an established treatment in second-line or salvage settings and is being investigated as a primary therapeutic alternative (Coleman 2002). For metastatic colorectal cancer, irinotecan was first used in salvage and evaluated in polytherapy settings (Hobday 2002) and is now used in first line therapy with fluorouracil, leucovorin, and oxaliplatin (Grothey 2004).

This class of drug specifically targets topoisomerase I (top1), a DNA unwinding enzyme responsible for relaxation of DNA during replication and transcription, as well as other functions including DNA repair and recombination. Top1 is also a kinase that participates in RNA processing.

Top1's varied activities situate it in several protein complexes and in physical association with many proteins including TATA-binding protein (TBP), p14ARF, PSF/p54, and p53 (Mao 2002: Karayan 2001; Straub 1998). Top1 has been detected in RNA polymerase (RP) I, II and III transcription complexes (Hannan 1999; Desai 2003; Rose 1988; Wang 1998) and in replication complexes (Lebel 1999; Loor 1997). Upon DNA damage, nucleolar top1 relocalizes (Thielmann 1999) and associates with p53 where it may function in DNA repair processes. Ubiquitination and proteosome degradation play important roles in the removal of top1-DNA complexes and can play a role in cellular resistance to CPT (Desai 2001). Poly ADP ribosylation inhibits top1 activity (Bauer 2000; Smith 1999), while phosphorylation by at least two kinases has been demonstrated to increase top1 activity (Pommier 1990; Yu 2004). Top1 is also known to bind Werner syndrome helicase, a protein involved in recombinational repair and replication (Lebel 1999). Additionally, top1 controls transcription of some genes via interaction with cis-acting regulatory gene elements and regulates transcript processing via its phosphorylation of, and association with, serine/arginine rich RNA splicing factors (Rossi 1996; Merino 1993).

The major toxicity of the CPT drugs can arise during S phase by stabilization of the covalent top1-DNA nucleoprotein complex while a single stranded scission is present in the DNA (Hsiang 1989). This is termed the cleavable complex (Hsiang 1989). Double stranded DNA breaks result from replication runoff when this complex is encountered on the leading strand of DNA synthesis by the cells' replication machinery (Strumberg 2000), and these can account for the major cytotoxic effects of the drug. Another cause of cytotoxicity can be that CPT poisoning induces apoptosis by targeting telomeric repeats that have multiple copies of the top1 cleavage sequence 5′-TT ↓ AGGG-3′ (downward arrow denotes site of scission) (Kang 2004). What is needed in the art are peptides that interact with topoisomerase I and have minimal cytotoxic effects.

### III. SUMMARY

Disclosed herein are isolated peptides comprising an amino acid sequence selected from the group consisting of: SEQ ID NOS: 1, 3-7, or 20-28, an amino acid sequence at least about 90% identical to the amino acid sequence of SEQ ID NOS: 1, 3-7, or 20-28, or the amino acid sequence of SEQ ID NOS: 1, 3-7, or 20-28 having one or more conservative amino acid substitutions.

Also disclosed are methods of treating a disease associated with topoisomerase I, the method comprising: identifying a subject having a disease associated with the topoisomerase I; and administering to the subject a composition comprising the peptides disclosed herein.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an analytical titering of phage from top1-DNA-9AC biopanning screen using only top1 as substrate.

FIG. 2 shows A) Biosensor data generated from several concentrations of top1 injected over the T1BP2 surface. These top1 experiments were evaluated with BIAevaluation Software 3.1 using Langmuir Kinetics to model the interaction. The accuracy of the fit to the model is illustrated by dashed lines representing actual data and narrow lines representing the fitted curves. B) The data indicate a high nanomolar affinity (K_{D}) of top1 for the novel peptide ligand. C) Shows biosensor response (summarized from three experiments) to the injection of 8.0 nM top1, topoisomerase II, and tubulin over the channel derivatized with the T1BP2.

FIG. 3 shows A) 100 kDa top1-DNA relaxation gels with (lower) and without (upper) 1 μM T1BP2. Inhibition of top1 relaxation (slight increase in supercoiled DNA remaining) by T1BP2 activity was observed in lanes 1 and 2, which had relatively high concentrations of top1. B) Cleavage complex assays containing T1BP2: Lane 1 the DNA substrate only (note that there is some endogenous nicked DNA). In lanes 2-12, 20 ng of top1 and the indicated amounts of peptide and 9AC were added to each reaction. At 0.45 μM 9AC, the addition of peptide caused an increase in cleavage at all but one concentration in this experiment.

FIG. 4 shows cytotoxicity of T1BP2-T on HCT 116 colon cancer and MDR cells. A) 0.005-50 μg/ml 9AC (▪), +50 μg/ml T1BP2-T ( [symbol] ) in HCT 116. B) 0.5 μg/ml 9AC+0.01-100 μg/ml T1BP2-T in HCT 116. C) 0.005-50 μg/ml doxorubicin (▪), +50 μg/ml T1BP2-T ( [symbol] ) in HCT 116. D) 0.05-50 μg/ml doxorubicin (▪), +50 μg/ml T1BP2-T ( [symbol] ) in A2780 ovarian cells MDR+; 0.05-50 μg/ml doxorubicin (•) in A2780 ovarian cells MDR−.

FIG. 5 shows the results of in vivo experiments. A) ♦, control HCT 116 tumor growth; ▪, 8 mg/kg 9AC; ▴, 8 mg/kg 9AC+T1BP2-T; ∘, 3 mg/kg 9AC; and ×, 3 mg/kg 9AC+T1BP2-T. At all doses tested, 9AC was found to have significant anti-tumor effects. Animals treated with 8 mg/kg 9AC and T1BP2-T had significantly lower (p<0.05) tumor growth than animals treated with 8 mg/kg 9AC alone (day 15). B) ♦, control HCT 116 tumor growth; ▴, 9 mg/kg 9AC; and 9 mg/kg 9AC+T1BP2-T. Tumor growth in T1BP2-T treated animals (p<0.05) was observed at days 12, 18, 21, and 26, compared to mice receiving 9AC alone.

FIG. 6 shows T1BP2 Similarities to top1 interacting proteins (those proteins that have been found in complexes with top1, immunoprecipitate with top1, found to bind top1 or affect top1 function in vitro).

FIG. 7 shows sequence alignments of reported top 1 interacting proteins with discovered pepitides CPP1 and CPP4.

FIG. 8 shows BIAcore analysis of 100 kDa top 1 association with CPP1 (left) and CPP4 (right). Top 1 was applied to flow cells in 1, 2.5, 5, 10 and 20 nM concentrations. The overlaying curves represent the actual data for a given concentration of top 1 and the “fit” curves. Data were fit using the BIAeval 3000 global fitting software.

FIG. 9 shows the effect of CPP4 on top 1-DNA binding. Biotinylated linear DNA was attached to the flow cell via straptavidin. Top 1 (5, 10 and 20 nM) was applied. Left shows top 1 dissociation in absence of CPP4, Right shows dissociation top 1 in the presence of 50 nM CPP4, dissociation is slowed.

FIG. 10 shows bia-data supporting phage library enrichment. It is a composite of two sensorgrams from biosensor analysis of amplified second round of top1-suicide substrate affinity selected phage. The upper and lower traces are of an injection of second round of the biopanned phage passing over differently derivatized channels. The surface of the channel 1 (upper curve) is streptavidin-anchored biotinylated double-stranded DNA. The surface of the bottom trace is biotin-blocked streptavidin. First, top1 is injected over the chip (injection not shown) and it binds only to the DNA coated lane (upper curve). The decrease in resonance units (RU) in the upper trace from 0-50 seconds is part of the top1-DNA dissociation curve. Top1-affinity selected phage were then injected at 50 seconds and found to bind to the lane with top1-DNA, but not significantly to the lane without top1 (lower curve). Control experiments showed that unselected phage did not bind the top1-coated surface and DNA alone was insufficient to bind phage.

FIG. 11 shows fluorescence polarization analysis of topoisomerase I-peptide binding. Data was acquired with human placental 91 kDa top1 in 150 mM HBS, 1 mM DTT at room temperature. Top1 was titered into 500 nM fluorescein labeled T1BP1. The excitation wavelength was set at 490 nM filtered at 495 nm. Emissions were collected between 500 nm and 540 nm with maximum at 520 nm. The y axis mP is defined as the dimensionless milli-polarization unit.

FIG. 12 shows Top 1 catalysis assays. Left shows: lanes 2, 3 & 4 DNA and top 1 (500 μM, 100 μM & 50 μM respectively); 5, 6 & 7 DNA and top 1 (500 μM, 100 μM & 50 μM respectively) and 500 μM CPP1; 8, 9 & 10 like 5, 6 & 7 except 50 μM CPP1 Right shows: lane 1 DNA in reaction buffer only; column 2, 3 & 4 DNA and top 1 (500 μM, 100 μM & 50 μM respectively); 5, 6 & 7 DNA and top 1 (500 μM, 100 μM & 50 μM respectively) and 50 μM CPP4; 8, 9 & 10 like 5, 6 & 7 except 50 μM CPP4 followed by proteinase K digestion.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. General

Top1 was examined using phage display to identify top1-binding peptide ligands capable of binding top1 with high affinity. Then, because of the significance of the CPT class of drugs and the myriad of top1-protein interactions known, the possibility that these novel top1-binding peptides have pharmacological effects was explored. It was hypothesized that top1 interacts with proteins via epitopes formed by topographically clustered amino acid sequences displayed on its surfaces, as it does with DNA (Redinbo 1998). Short peptides that bind such epitopes can mimic a subset of the total protein-protein interactions and act as agonists or antagonists of select top1 activities. It was hypothesized that the ability to selectively interfere with, or mimic, specific sites of protein-top1 interaction can yield new top1 directed therapeutics. Several peptides with high affinity for top1 were discovered and these were examined for top1 affinity, top1 catalytic and cleavage complex effects and for cytotoxic effects in cultured cell lines. Although several peptides exhibited nanomolar affinity for top1, none had cytotoxic effects when administered alone. However, in combination with 9-amino camptothecin (9AC), one TAT labeled-15mer peptide had synergistic cytotoxic effects with 9AC both in the cytotoxicity assay and in a nude mouse xenograft human tumor model.

Therefore, disclosed herein are peptides that are capable of acting synergistically with a chemotherapeutic agent in the treatment of cancer. For example, those peptides disclosed in SEQ ID NOS: 1, 3-7, or 20-28 have been found to have this ability.

The use of a combination of therapeutic agents is common in the treatment of neoplastic diseases. For example, paclitaxel (Taxol™) has been approved by the U.S. FDA for use with cisplatin in the treatment of ovarian carcinoma. U.S. Pat. No. 5,908,835 to Bissery et al. claims synergy of using paclitaxel or docetaxel in combination with an anthracycline antibiotic such as daunorubicin or doxorubicin. Similarly, U.S. Pat. No. 5,728,687 to Bisser et al. Claims synergy of using paclitaxel or docetaxel in combination with an alkylating agent, epidophyllotoxin, antimetabolite, or vinca alkaloid.

However, such combinations heretofore have not included the use of small peptides and in particular those disclosed herein.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.
1. Peptides
   a) Protein Variants

As discussed herein there are numerous variants of the peptides disclosed herein that are known and herein contemplated. Peptide variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations
TABLE 1: Amino Acid Abbreviations

| Amino | |
|---|---|
| Acid | Abbreviations |
| alanine | Ala (A) |
| allosoleucine | AIle |
| arginine | Arg (R) |
| asparagine | Asn (N) |
| aspartic acid | Asp (D) |
| cysteine | Cys (C) |
| glutamic acid | Glu (E) |
| glutamine | Gln (K) |
| glycine | Gly (G) |
| histidine | His (H) |
| isolelucine | Ile (I) |
| leucine | Leu (L) |
| lysine | Lys (K) |
| phenylalanine | Phe (F) |
| proline | Pro (P) |
| pyroglutamic acid | PGlu |
| serine | Ser (S} |
| threonine | Thr (T) |
| tyrosine | Tyr (Y) |
| tryptophan | Trp (W) |
| valine | Val (V_{—} |

TABLE 2
Amino Acid Substitutions
Original Residue Exemplary Conservative Substitutions,
others are known in the art.

Ala; ser
Arg; lys, gln
Asn; gln; his
Asp; glu
Cys; ser
Gln; asn, lys
Glu; asp
Gly; pro
His; asn; gln
Ile; leu; val
Leu; ile; val
Lys; arg; gln
Met; leu; ile
Phe; met; leu; tyr
Ser; thr
Thr; ser
Trp; tyr
Tyr; trp; phe
Val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed peptides herein is through defining the variants and derivatives in terms of homology/identity to the known sequences which are disclosed in SEQ ID NOS: 1-30. Specifically disclosed are variants of these and other proteins herein disclosed which have at least, 70% or 75% or 80% or 85% or 90% or 95% homology to the stated sequence. Those of skill in the art readily understand how to determine the homology of two peptides, and how to determine which changes to the peptide can be made while retaining the function of the peptide, in this case, its anti-tumor capability when used with camptothecin.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).
2. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).
3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.
4. Nucleic Acids

There are a variety of molecules disclosed herein that are nucleic acid based, including for example the nucleic acids that encode, for example, the peptides disclosed herein, as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.
a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.
b) Sequences

There are a variety of sequences related to, for example, topoisomerase I, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.
c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.
5. Delivery of Peptides/Combination Therapy

“Chemotherapeutic agents” are defined as agents that attack and kill cancer cells. They can be used alone or in combination with one or more other chemotherapeutic agent. Specifically, they can be used in combination with the peptides disclosed herein in SEQ ID NOS: 1, 3-7, or 20-28, and optionally with other chemotherapeutic agents as well.

Chemotherapeutic cancer agents include numerous compounds such as taxane compounds and derivatives. Although taxane compounds were initially extracted from the Pacific yew tree, Taxus brevifolia. They include, for example, paclitaxel and its derivatives or docetaxel and its derivatives. Additional taxane derivatives and methods of synthesis are disclosed in U.S. Pat. No. 6,191,287 to Holton et al., U.S. Pat. No. 5,705,508 to Ojima et al., U.S. Pat. Nos. 5,688,977 and 5,750,737 to Sisti et. al., U.S. Pat. No. 5,248,796 to Chen et al., U.S. Pat. No. 6,020,507 to Gibson et al., U.S. Pat. No. 5,908,835 to Bissery, all of which are incorporated by reference.

Some chemotherapeutic cancer agents are mitotic inhibitors (vinca alkaloids). These include vincristine, vinblastine, vindesine and Navelbine™ (vinorelbine, 5′-noranhydroblastine). Similarly, chemotherapeutic cancer agents include topoisomerase I inhibitors such as camptothecin compounds.

As used herein, “camptothecin compounds” include Camptosar™ (irinotecan HCL), Hycamtin™ (topotecan HCL) and other compounds derived from camptothecin and its analogues. Another category of chemotherapeutic cancer agents are podophyllotoxin derivatives such as etoposide, teniposide and mitopodozide.

Other chemotherapeutic cancer agents are alkylating agents, which alkylate the genetic material in tumor cells. These include cisplatin, cyclophosphamide, nitrogen mustard, trimethylene thiophosphoramide, carmustine, busulfan, chlorambucil, belustine, uracil mustard, chlomaphazin, and dacarbazine.

Additional chemotherapeutic cancer agents are antimetabolites for tumor cells. Examples of these types of agents include cytosine arabinoside, fluorouracil, methotrexate, mercaptopurine, azathioprime, and procarbazine.

An additional category of chemotherapeutic cancer agents includes antibiotics. Examples include doxorubicin, bleomycin, dactinomycin, daunorubicin, mithramycin, mitomycin, mytomycin C, and daunomycin. There are numerous liposomal formulations commercially available for these compounds. Also, other chemotherapeutic cancer agents include anti-tumor antibodies, dacarbazine, azacytidine, amsacrine, melphalan, ifosfamide and mitoxantrone.

As used herein, “enhancement” refers to a synergistic effect as determined from measurement of the enhanced factor, as defined below. In general, an enhanced factor of two or greater is considered synergistic while an enhanced factor greater than one may be synergistic. For example, if one of the compounds has little individual chemotherapeutic effect, an enhanced factor greater than one indicates a synergistic effect is occurring.

In one example of treating cancer, one of the peptides disclosed herein, namely SEQ ID NO: 1, 3-7, or 20-28, and a chemotherapeutic cancer agent are administered to the patient. The combination therapy enhances the effect of the chemotherapeutic cancer agent and prevents multi-drug resistance from developing. Examples include, without limitation, taxane compounds, vinca alkaloids, camptothecins and antibiotics useful as chemotherapeutic agents.

The peptides can be administered in a time-release manner when permitted by the chemotherapeutic agent. Suitable time-release devices are well known to those of skill in the art. For example, the time-release effect may be obtained by capsule materials that dissolve at different pH values, by capsules that release slowly by osmotic pressure, or by any other known means of controlled release. U.S. Pat. No. 6,306,406 to Deluca discloses a number of time-release methods and related references, the contents of which is incorporated herein.

The peptides disclosed herein (also referred to herein as “compositions”) can be administered in vivo in a pharmaceutically acceptable carrier. By “pharmaceutically acceptable” is meant a material that is not biologically or otherwise undesirable, i.e., the material may be administered to a subject, along with a chemotherapeutic agent, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The peptides, which can be comprised in a composition, can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the peptides, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).
a) Pharmaceutically Acceptable Carriers

The peptides can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.
b) Therapeutic Uses

Effective dosages and schedules for administering the peptides may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed peptide for treating, inhibiting, or preventing cancer, the efficacy of the peptide or peptides can be assessed in various ways well known to the skilled practitioner. For instance, one of ordinary skill in the art will understand that a composition, such as a peptide, disclosed herein is efficacious in treating or inhibiting cancer in a subject by observing that the peptide enhances the therapeutic usefulness of a chemotherapeutic agent.
6. Compositions Identified by Screening with Disclosed Compositions/Combinatorial Chemistry
   a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed in SEQ ID NOS: 1-30 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, topoisomerase I, are also disclosed. Thus, the products produced using the combinatorial or screening approaches disclosed herein also considered herein disclosed.

It is understood that the disclosed methods for identifying peptides that act synergistically with chemotherapeutic agents can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

There are a number of methods for isolating peptides which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating peptides that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.
b) Computer Assisted Drug Design

The disclosed compositions can be used as targets for any molecular modeling technique to identify either the structure of the disclosed compositions or to identify potential or actual molecules, such as peptides, which interact in a desired way with topoisomerase I

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as peptides, are also disclosed. Thus, the products produced using the molecular modeling approaches that involve the disclosed compositions, such as peptides also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.
7. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include the peptides and derivative thereof disclosed herein, as well as a chemotherapeutic agent to be used in combination with the peptide. For example, disclosed is a kit for treating cancer comprising any one or more of SEQ ID NO: 1, 3-7, or 20-28 or a derivative thereof, and a chemotherapeutic agent such as, for example, camptothecin.

D. Methods of Making the Compositions

The compositions disclosed herein and the compositions necessary to perform the disclosed methods can be made using any method known to those of skill in the art for that particular reagent or compound unless otherwise specifically noted.
1. Peptide Synthesis

One method of producing the disclosed proteins, such as those found in SEQ ID NOS: 1-30, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

E. Methods of Using the Compositions
1. Methods of Treating Cancer

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

The peptides and chemotherapeutic agents disclosed herein may also be used for the treatment of precancer conditions such as cervical and anal dysplasias, other dysplasias, severe dysplasias, hyperplasias, atypical hyperplasias, and neoplasias.

F. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Example 1
a) Methods

Isolation of human top1: Human top 1 was obtained both from baculovirus-expressing insect cells and human placenta. Sf9 Insect cells were grown in one liter cultures, infected with baculovirus stock and isolated by centrifugation 50 hrs later (Stewart 1999). Top1 from the sf9 cells or from placenta was isolated and purified utilizing the method of Holden et al. (Holden 1990). Successful isolation of top1 was confirmed by DNA relaxation activity assays and SDS PAGE/Western analysis

Phage display: Phage display was performed as described by Smith (Parmley 1989). Briefly, top1 was first biotinylated with a biotin derivative containing a spacer engineered to reduced steric interference (Pierce EZ-Link Biotin-PEO-Amine). The reaction was performed at a 5:1 molar ratio of biotin:top1 in 10 mM HEPES (pH 8.2) buffered 500 mM NaCl. The unreacted biotin was removed by gel filtration in a Biospin 30 column (BioRad) and biotinylated top1 was assayed for activity using a pull down method with streptavidin coated beads. The biotinylated top1 maintained catalytic activity. For phage display, 20 μg biotinylated top1 was reacted with a mixture of pBR322 and 9AC and 20 μg was applied to a 30 mm plastic petri dish coated with 10 μg of neutravidin (Pierce). The dish was then blocked with 3.0% bovine serum albumin, and 10 μl of a 10⁹/μl titer phage library in 100 μl TBS-0.05% Tween was added. Phage were allowed to associate with gentle rocking for 2 hours at 4° C., and then washed repeatedly over 2 hours at 4° C. with buffered saline containing 0.1% Tween. The bound phage were then eluted in 0.1 N HCl-glycine, and used to infect competent K91 Kan E. coli and amplified. Phage were harvested, concentrated by PEG precipitation, titered, amplified, and the process was repeated. After 2-3 rounds of screening, phage clones were picked and their DNA sequenced to determine the displayed peptides.

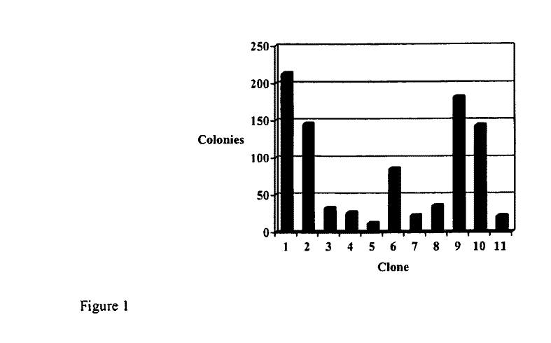

Analytical titering: Ten μl of amplified phage clones (approximately 10¹² cfu) were incubated in 50 nM top1 for one hour at 4° C. on a rocker. These reactions were then applied to BSA-blocked, streptavidin-coated plates. The plates were rocked while incubated at 4° C. for another hour and then washed five times with TBS/Tween. Competent E. coli were added directly to the washed plates and the output assessed by counting a 10⁻⁶ dilution of the culture on tetracycline plates (FIG. 1). Column 11 of FIG. 1 presents the control experiment titering of unselected phage library.

Peptide synthesis: For relaxation assays and peptide affinity studies the peptides were synthesized as sequenced from the phage. For the cell and mouse studies a domain that enhances cellular uptake (referred to as a TAT domain and signified as -T) was added with short spacer (alanine-glycine) to the C-terminus of the peptide (Vives 1997). Peptides not containing the added TAT domain had no detectable effects on cells in culture.

Sequence analysis: Similarity searches were performed using BLAST at NCBI (http://www.ncbi.nlm.nih.gov/blast) in the Swiss-Prot database of protein sequences (Swiss-Prot Release 44.5 of 13 Sep. 2004 at http://www.expasy.org/sprot). Only human sequences were used in the similarity analysis. With the exception of P54/nrb, all the top1-binding proteins referred to in this work are in the Swiss-Prot sequences. Sequence analysis (identification of top1 binding proteins with similar sequence) was performed using the BLAST algorithm and the PAM 30 matrix, word size of two, expect value of 20000 out of the entire Swissp-Prot atabase.

Binding analysis: The binding analysis of the peptides was performed on a BIAcore 2000 instrument. A four channel CM5 sensor chip was derivatized with three top1 binding peptides (T1BPs 1, 2 and 4) and a control surface left blank. Catalytically active top1 was used as the mobile or analyte phase and diluted in 150 mM NaCl, 30 mM HEPES, 0.05% P20 and injected at various concentrations (8.0, 4.0, 2.0, 1.0 and 0.4 nM) through the instrument's microfluidics sample handling system at a flow rate of 30 μl/minute. With each injection of a particular concentration of top1, association and dissociation data for three peptides were generated. Binding constants were determined using the BIAevaluation 2.1 software.

Top1 relaxation/cleavage activity assays: Peptides were assayed for their ability to interfere with or enhance relaxation of supercoiled DNA mediated by 100 kDa top1. The top1 and pBR322 were combined in a top1-reaction buffer (150 mM KCl, 12 mM MgCl₂, 2 mM EDTA, 50 mM Tris HCl pH 7.5) at 37° C. for 30 minutes with 1 μM peptide. The 20 μl reactions were stopped with 1.0 μl of 1% SDS. Digestion of protein was performed by addition of 1.0 μg proteinase K for 30 minutes at 37° C., loaded with 5 μl of a standard 5×bromphenol blue/glycerol loading buffer and run in an 0.8% agarose gel in TAE (0.04 M Tris-acetate and 1.0 mM EDTA). DNA cleavage assays were performed in 20 μl volumes of reaction buffer (50 mM Tris-HCl (pH 7.5), 100 mM KCl, 10 mM MgCl₂ 0.5 mM EDTA, 0.2 mg/ml BSA), 500 ng of radiaolabeled (³H thymidine) supercoiled rf (replicative form) M13 mp 19 DNA and 112 ng of top1. The reactions were incubated for 30 minutes at 30° C. and then treated with 1 μl of 1 mg/ml proteinase K in 0.05% SDS for an additional 30 minutes at 37° C. to transform the 9AC-top1-DNA complexes into nicked DNA. This was followed by electrophoresis in an 0.8% agarose-TAE gel containing ethidium bromide. A control set of experiments and log serial dilutions of peptide were examined for each peptide. Percent cleavage was determined by liquid scintillation counting of excised bands using the method described by Marshall et al. (Marshall 2003).

Cell localization study: Part of each batch of peptide synthesized with the TAT domain was labeled with an immuno-fluorescent dye (FITC) and confocal microscopy experiments confirmed peptide uptake into live cells. HCT 116 (human colon tumor cells) were grown in McCoy's medium supplemented with 7.5% calf serum/ 2.5% fetal calf serum (Atlanta Biologicals), 10.0 units/ml penicillin and 100 μg/liter streptomycin. Cells grown to 30% confluence in 60 mm plates were washed in PBS and the labeled TAT linked peptide was diluted to 1 μM and added to the plate in unsupplemented McCoys medium and allowed to incubate at 37° C. for 1 hour. The plates were washed in PBS, supplemented medium reapplied and the cells visualized.

Cytotoxicity assay: HCT 116 cells grown as monolayer cultures (as above) were harvested by trypsinization prior to confluence and seeded at 20,000 cells per well in 200 μl corning 96 well microtiter plates in the same medium. Cell viability was determined at three days using the MIT assay as described by Marshall and coworkers adapted from Mosmann et al. (Marshall 2003, Mosmann 1983). A2780 ovarian MDR+ and MDR− were obtained and grown as the HCT 116 cells except in supplemented α-MEM.

In vivo mouse studies: HCT 116 cells (4×10⁶) were injected into the flanks of nude (BalbC nu/nu) mice for both experiments. The animals were randomized when the tumors were staged at 50 mm³. In the first experiment, control animals were administered 0.1 ml of vehicle (α-MEM containing 0.05% methylcarboxycellulose and 2% DMSO) i.p., in the absence of peptide (PBS, control #1) or presence of T1BP2-T (control #2, 150 μg in 0.1 ml PBS) or T1BP3-T (control #3, 150 μg in 0.1 ml PBS) injected s.c. at site of tumor. No difference in tumor growth was observed for these control groups and the results were combined as control (n=15). Animals treated with 9AC were divided into 4 groups, each containing 5 mice. T1BP-T and PBS subcutaneous injections were administered as described above at the site of the tumor, 30 minutes after the i.p. injection of 9AC in a 50 μl volume. 9AC was dissolved in vehicle. Groups consisted of animals treated with a total of 4 mg/kg 9AC (low dose) in the absence or presence of T1BP2-T or a total of 8 mg/kg 9AC (high dose). Animals were treated with 1 or 2 mg/kg doses, twice a week over 2 weeks.

In the second experiment, 18 mice were evaluated in three groups of six animals. Control animals were injected i.p. with 0.1 ml of vehicle (α-MEM containing 0.05% methylcarboxycellulose and 2% DMSO). Treated mice received a total of 9 mg/kg 9AC, 3 mg/kg in 0.1 ml vehicle a week over 3 weeks, in the absence or presence of 150 μg T1BP2-T in 0.1 ml PBS injected s.c. at the site of the tumor 30 min after 9AC injection. Animals were sacrificed when their tumors exceeded 15% of their body weight. IACUC approval #UU 00-05004.
b) Results

Phage display: During each successive round of the biopanning process, the relative affinity of the phage for the top1 target complex were assessed by comparing the output of the top1-DNA-9AC plate or a control plate coated with Neutravidin and blocked with BSA. Approximately ten fold more phage were recovered from the top1-DNA-9AC coated plate than from the control plate after the third round. Since this output probably had phage that bound both DNA and top1, several clones from the third round were sequenced and subjected to analytical titering against top1 alone. Several examples of the results are presented in FIG. 1. These binding affinity, as represented by colonies recovered form the tittering experiment were quite dissimilar, as were the displayed peptide sequences. From the results of this experiment, several of the phage displayed peptides were synthesized and subjected to biophysical and biological assays to assess top1 binding affinity and in vitro and in vivo activity. These short peptide sequences were also queried for identities to known top1-binding proteins by BLAST searches, Table 1. This report focuses on the one peptide that had both in vitro and in vivo activity, SAYAATVRGPLSSAS (T1BP2, SEQ ID NO: 1).

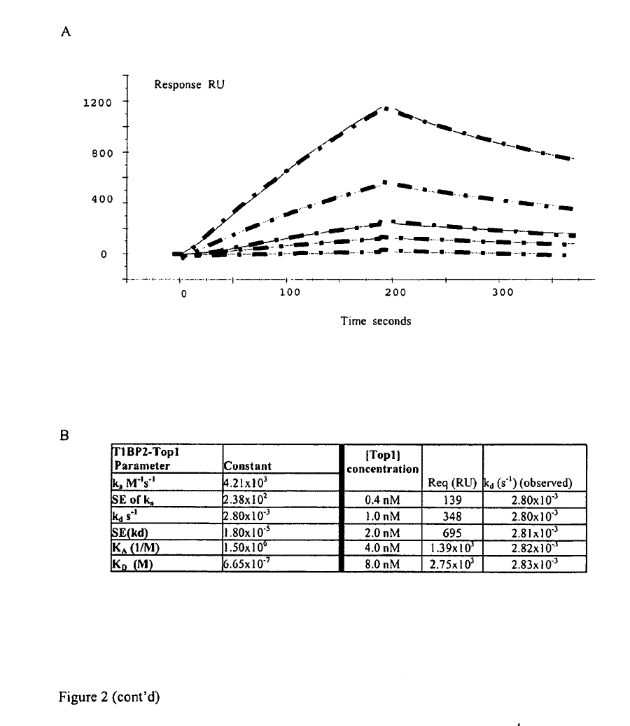

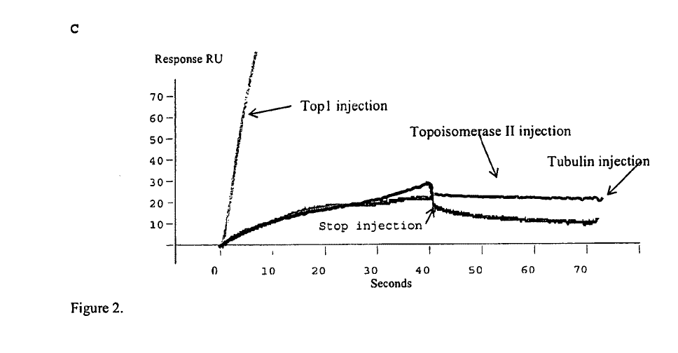

T1BP2 affinity for top1: T1BP2 had remarkable affinity for top1 when assayed by surface plasmon resonance (FIG. 2a). When the kinetics of this interaction was modeled using the Langmuir model of association and dissociation, the K_{D}) was determined to be 6.65×10⁻⁷ M. FIG. 2b illustrated that the model fits the data well; the association and dissociation curves that fit the peptide-top1 data were coincidental with curves generated by the model.

T1BP2 binding specificity for top1: The biosensor surface, derivatized with T1BP2, was also used to quickly and efficiently evaluate the specificity of the peptide for top1 verses topoisomerase II, a functional analog of top1, and tubulin, a protein unrelated to top1 but known to interact with many other proteins. The traces in FIG. 2c confirm the peptide's specificity for top1 in the context of these two proteins.

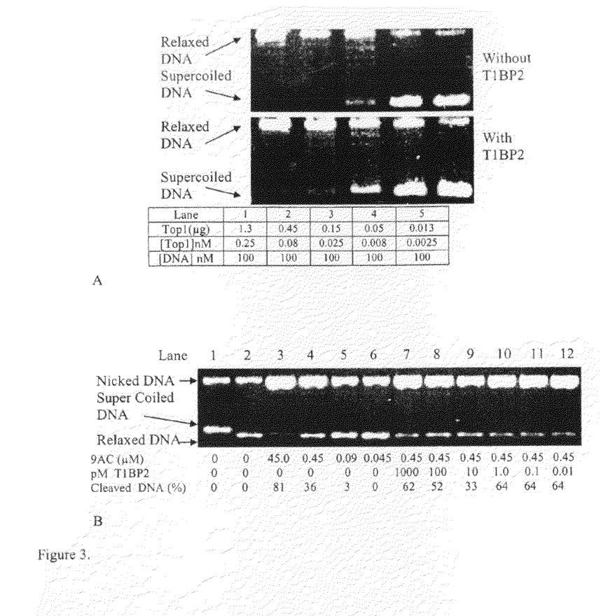

T1BP2 effects on top1 -mediated DNA relaxation and cleavage complex formation: FIG. 3a is an image of a DNA relaxation assay run in the presence of T1BP2. Although other peptides discovered in our biopanning process had the ability to moderately interfere with or enhance the activity of top1, T1BP2 had a slight ability to inhibit top1-mediated relaxation. In the cleavage complex assay (FIG. 3b), T1BP2 enhanced the formation of cleavage complexes at intermediate concentrations of 9AC (data from 0.45 μM and 0.9 μM; 0.45 μM data shown). At these concentrations of 9AC, through the range of peptide concentrations tested, T1BP2 increased cleavage complex formation approximately 50%.

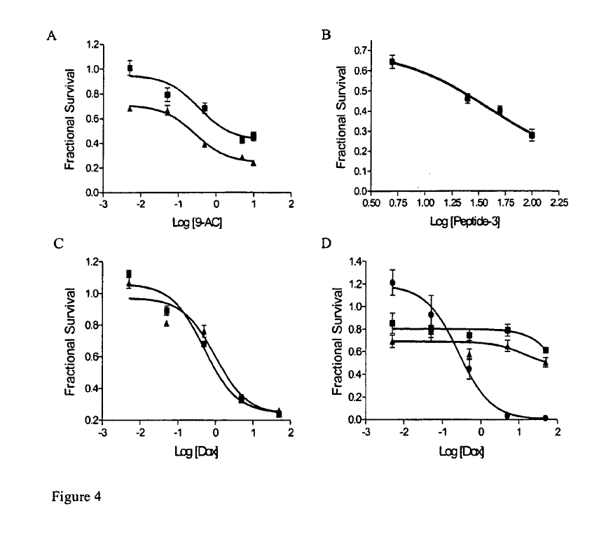

Cellular toxicity studies: All five peptides were also assayed for toxicity in tumor cell lines. The cellular toxicity data presented in FIG. 4 reveal that T1BP2-T had no toxicity when used alone (data not shown), but acted synergistically when added with 9AC. The effect of T1BP2-T appears to be specific to top1 poisons because it did not significantly enhance the cytotoxicity of doxorubicin (FIGS. 4c and 4d), etoposide or UV light in HCT 116 cells. The selective synergism with 9AC showed that T1BP2-T is a top1 drug-sensitizer with the potential for improving human cancer chemotherapy. Experiments with multidrug resistant and normal ovarian cancer cells showed that drug sensitization is not likely due to MDR-mediated effects.

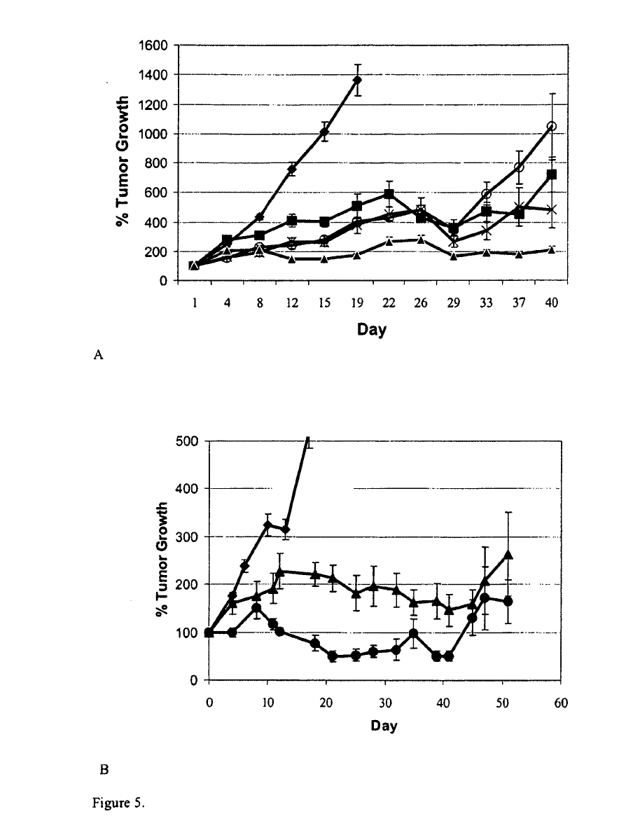

In vivo activity of T1BP2 in nude mice: Nude mice bearing tumors derived from cultured HCT 116 cells and receiving 9AC chemotherapy were administered T1BP2-T. In two experiments, graphed in FIG. 5, the in vivo activity of T1BP2-T corresponded with the in vitro effects in the cleavage assay, that is, it increased the activity of 9AC. In the first experiment, T1BP2-T had no demonstrable effect in animals treated with 1 mg/kg 9AC on days 1, 4 and 8 (3 mg/kg 9AC). However, in animals treated with higher concentrations of 9AC, T1BP2-T had a statistically significant p<0.05 augmentation of 9AC's antitumor effect (day 15). The second experiment was similar except that mice received 3 mg/kg 9AC injections, with or without T1BP2-T, on days 1, 8 and 18. Significantly improved (p<0.05) response was observed on days 12, 18, 21 and 26.
c) Discussion

Low micromolar concentrations of T1BP2 had the ability to alter the in vitro catalytic rate of top1-mediated DNA relaxation. It also had the ability to increase the amount of cleaved DNA at certain 9AC-top1 concentrations. Most significantly, although nontoxic when administered alone, T1BP2 sensitized tumor cells to 9AC, both in cellular based assays and in a mouse tumor model. T1BP2 increased top1-mediated DNA cleavage complex formation in vitro and the in vivo effect of T1BP2 significantly increased drug-specific killing of tumor cells.

Specific killing of tumor cells by 9AC is thought to be primarily due to DNA double strand breaks (DSB) produced during DNA replication in S-phase. Therefore, the most direct explanation of the increased killing of the tumor cells with the combination of T1BP2 and 9AC is that T1BP2 stabilizes top1-DNA-9AC and this leads to more DSB. This is consistent with the DNA cleavage assay results, but does not rule out other potential mechanisms of sensitization.

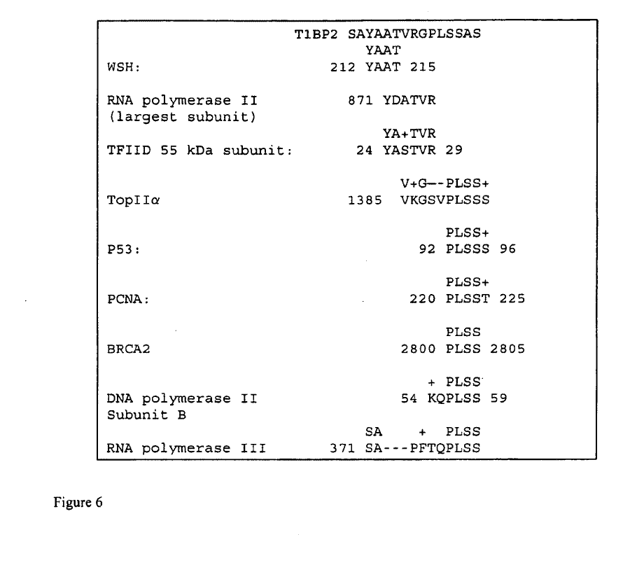

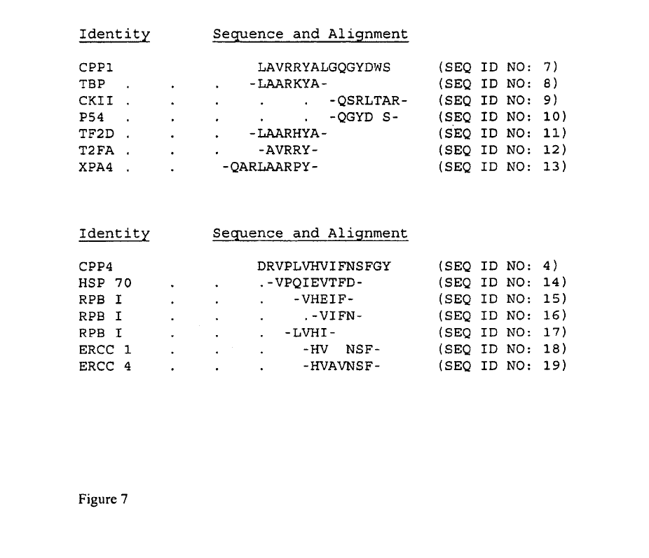

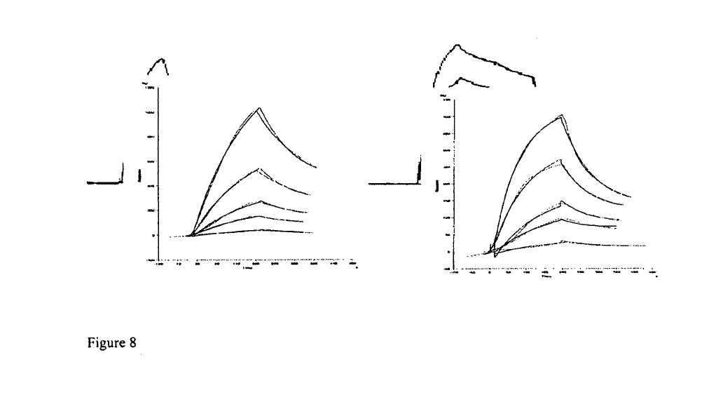

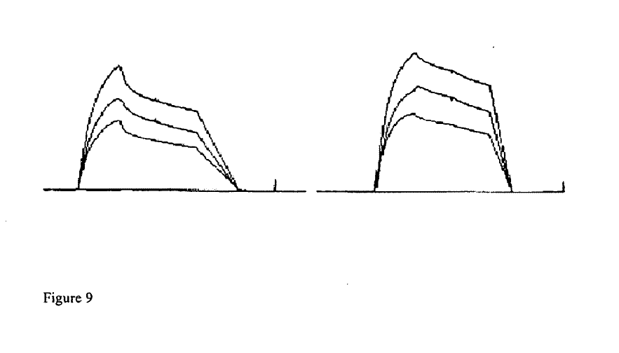

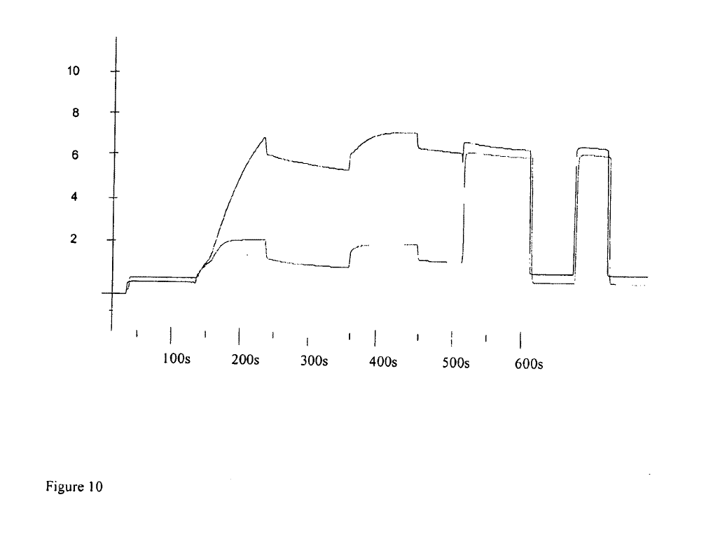

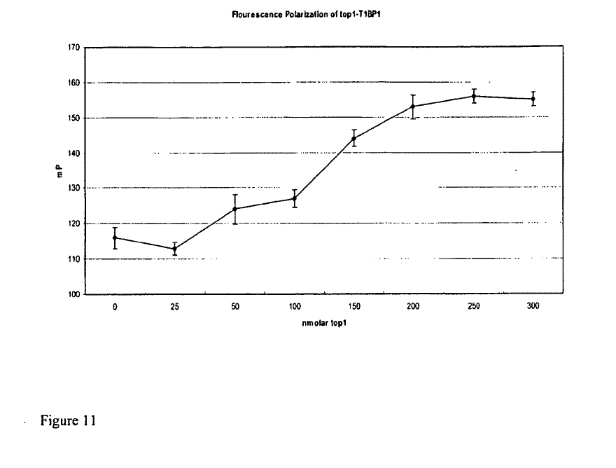

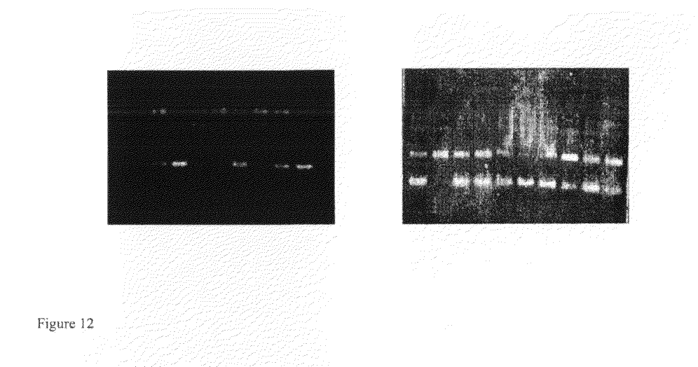

When the Swiss-Prot database was queried with six residue fragments of the peptide sequence, several interesting and high similarity matches were found. FIG. 6 illustrates these identities. The N terminus of the peptide (YAATDR, SEQ ID NO: 2) was found to align with RNA pol II (largest subunit), TFIID 55 (found in transcriptional complexes with top1, and Werner syndrome helicase, a top1 and p53 binding protein (Blander 1999). The C-terminal sequence of the peptide has similarity to p53, BRCA2, DNA polymerase II B, topIIα, and DNA and RNA polymerases; all of which have been found in complexes with top 1, demonstrated to interact with top1 and/or be modulated by top1 (Marshall 2003; Mosmann 1983; Carty 2002; Czubaty 2005; Gobert 1996). The PLSS sequence, though short and not unique, is found in only about 1/200 proteins in the Swiss-Prot human database. YAATDR (SEQ ID NO: 2) was found less frequently.

This sequence similarity information suggests several signaling-pathway dependent mechanisms that could cause sensitization of cancer cells to killing by 9AC. Upon induction of ionizing radiation induced DSB lesions in cells, top1 is very quickly delocalized from the nucleolus, ribosylated, complexed with p53 and relocalized at discrete foci. Although PLSS is not in the reported binding domain of p53 for top1 (reported to be p53 residues 302-321 (35)), the PLSS region has been implicated in SH3 mediated interactions functioning in base excision repair of DNA damage and p53-mediated apoptosis in response to gentoxic stress (Jiang 2001). It is also a site of mutational activation of p53 (DeVries 1996). The similarity of T1BP2 to p53 suggests other possible mechanisms of cell sensitization. Along with other proteins, p53 helps to activate the two major DNA damage-dependent cellular checkpoints, the G₁/S and the G₂/M, as well as direct apoptosis. These mechanisms respectively allow DNA repair or commit cells to death, depending on the severity, type and cell cycle timing of DNA damage. Tumor cell killing can be attributed to increased sensitivity to apoptotic stimuli or attempted replication of a severely damaged genome in cells exposed to a p53 mimic peptide. Additionally, given the potential lethality of top1 lesions on DNA and the economy of top1 demanded by its various cellular roles, top1 access to DNA at potential cleavage complex sites can be an important factor. If p53 simply sequesters top1 to reduce potential replication fork induced DSBs; interruption of the p53-top1 interaction can cause the cell killing effects observed.

BRCA2, a scaffolding protein also involved in DNA repair, has not been demonstrated to directly interact with top1 protein but a recent study found BRCA2 to increase top1 activity in cell extracts and modulate top1 mediated sensitivity to CPT (Rahden-Staron 2003).

In summary, this work describes the discovery of a short peptide, T1PB2, that binds top1 with mid-nanomolar affinity, inhibits top1 DNA relaxation, increases top1 mediated cleavage complex formation, increases tumor sensitivity to 9AC. The peptide also has interesting sequence similarity to several critical top1-binding DNA metabolizing enzymes, which, can explain the observed sensitization to 9AC.

TABLE 3
Examples of sequences of phage display derived
peptides that bind top1. T1BP1 through T1BP5
were isolated from the third round of biopanning
against a top1-DNA-9AC complex. EGQFTFPRGASE was
truncated due to a stop codon.

| | | Number of times |
|---|---|---|
| | | appeared/number of |
| Peptide Name | Sequence | cloned sequences |
| T1BP1 3rd round | SSQVVGVPQLMQSSP, | 1/5 |
| Top1-DNA-9AC | SEQ ID NO: 3 | |
| T1BP2 3rd round | SAYAATVRGPLSSAS, | 1/5 |
| Top1-DNA-9AC | SEQ ID NO: 1 | |
| T1BP3 3rd round | DRVPLVHVIFNSFGY, | 1/5 |
| Top1-DNA-9AC | SEQ ID NO: 4 | |
| T1BP4 3rd round | RNQGPVKMVFPIAPS, | 1/5 |
| Top1-DNA-9AC | SEQ ID NO: 5 | |
| T1BP5 3rd round | EGQFTFPRGASE*, | 1/5 |
| Top1-DNA-9AC | SEQ ID NO: 6 | |

TABLE 4
After 6 rounds of screening, 15 phage clones were
picked and their DNA sequenced used to determine
the displayed peptides. Sequence analysis showed
significant similarity (10/15 identity) between
CPP1 and a conserved domain RPII
(LAVRRYLAGQGY-DWS, Ref #125, SEQ ID NO: 7). CPP6
had a significant homology (6/7 identity) with
CCAAT (SEQ ID NO: 29)/enhancer binding protein
alpha (FTFPRGA, Ref. #126, SEQ ID NO: 30).

| CPP# | Amino Acid Sequence | SEQ ID NO: |
|---|---|---|
| 1 | L A V R R Y A L G N G Y D W S | 7 |
| 2 | S S Q V V G Y P Q L M Q S S P | 3 |
| 3 | S A Y A A T V R G P L S S A S | 1 |
| 4 | D R V P L V H V I F N S F G Y | 4 |
| 5 | R N Q G P V K M V F P I A P S | 5 |
| 6 | E G Q F T F P R G A S E | 6 |
| 7 | A V L V S L G G F S R A I P | 20 |
| 8 | A A V V P A H E V R F V P G S | 21 |
| 9 | S W S L G L G W P G H T S R G | 22 |
| 10 | P F A R A P V E H H D V V G L | 23 |
| 11 | F V L V R D T F P S S V C C P | 24 |
| 12 | R V P P R Y H A K I S P M V K | 25 |
| 13 | G Q R S S L A V V R Y S S G A | 26 |
| 14 | A E P S W V V S R Y H R S V V | 27 |
| 15 | R T W D S A L V P A N H V F V | 28 |

G. References
1. Coleman, R. L. Emerging role of topotecan in front-line treatment of carcinoma of the ovary. Oncologist, 7: 46-55, 2002.
2. Hobday, T. and Goldberg, R. Perspectives on the role of sequential or combination chemotherapy for first-line and salvage therapy in advanced colorectal cancer. Clin. Colorectal Cancer, 2: 161-169, 2002.
3. Grothey, A., Sargent, D., Goldberg, R. M., and Schmoll, H.-J. Survival of Patients With Advanced Colorectal Cancer Improves With the Availability of Fluorouracil-Leucovorin, Irinotecan, and Oxaliplatin in the Course of Treatment. J. Clin. Oncol., 22: 1209-1214, 2004.
4. Mao, Y., Mehl, I. R., and Muller, M. T. Subnuclear distribution of topoisomerase I is linked to ongoing transcription and p53 status. Proc. Natl. Acad. Sci. USA, 99: 1235-1240, 2002.
5. Karayan, L., Riou, J. F., Seite, P., Migeon, J., Cantereau, A., and Larsen, C. J. Human ARF protein interacts with topoisomerase I and stimulates its activity. Oncogene, 20: 836-848, 2001.
6. Straub, T., Grue, P., Uhse, A., Lisby, M., Knudsen, B. R., Tange, T. O., Westergaard, O., and Boege, F. The RNA-splicing Factor PSF/p54 Controls DNA-Topoisomerase I Activity by a Direct Interaction. J. Biol. Chem., 273: 26261-26264, 1998.
7. Hannan, R., Cavanaugh, A., Hempel, W., Moss, T., and Rothblum, L. Identification of a mammalian RNA polymerase I holoenzyme containing components of the DNA repair/replication system. Nucl. Acids Res., 27: 3720-3727, 1999.
8. Desai, S. D., Zhang, H., Rodriguez-Bauman, A., Yang, J.-M., Wu, X., Gounder, M. K., Rubin, E. H., and Liu, L. F. Transcription-dependent degradation of topoisomerase I-DNA covalent complexes. Mol. Cell Biol., 23: 2341-2350, 2003.
9. Rose, K. M., Szopa, J., Han, F. S., Cheng, Y. C., Richter, A., and Scheer, U. Association of DNA topoisomerase I and RNA polymerase I: a possible role for topoisomerase I in ribosomal gene transcription. Chromosoma, 96: 411-416, 1988.
10. Wang, Z. and Roeder, R. G. DNA topoisomerase I and PC4 can interact with human TFIIIC to promote both accurate termination and transcription reinitiating by RNA polymerase III. Mol. Cell, 1: 749-757, 1998.
11. Lebel, M., Spillare, E. A., Harris, C. C., and Leder, P. The Werner Syndrome Gene Product Co-purifies with the DNA Replication Complex and Interacts with PCNA and Topoisomerase I. J. Biol. Chem., 274: 37795-37799, 1999.
12. Loor, G., Zhang, S., Zhang, P., Toomey, N., and Lee, M. Identification of DNA replication and cell cycle proteins that interact with PCNA. Nucl. Acids Res., 25: 5041-5046, 1997.
13. Thielmann, H. W., Popanda, O., and Staab, H. J. Subnuclear distribution of DNA topoisomerase I and Bax protein in normal and xeroderma pigmentosum fibroblasts after irradiation with UV light and gamma rays or treatment with topotecan. J. Cancer Res. Clin. Oncol., 125: 193-208, 1999.
14. Desai, S. D., Li, T.-K., Rodriguez-Bauman, A., Rubin, E. H., and Liu, L. F. Ubiquitin/26S Proteasome-mediated Degradation of Topoisomerase I As a Resistance Mechanism to Camptothecin in Tumor Cells. Cancer Res., 61: 5926-5932, 2001.
15. Bauer, P. I., Buki, K. G., Comstock, J. A., and Kun, E. Activation of topoisomerase I by poly [ADP-ribose] polymerase. Int. J. Mol. Med., 5: 533-540, 2000.
16. Smith, H. M. and Grosovsky, A. J. PolyADP-ribose-mediated regulation of p53 complexed with topoisomerase I following ionizing radiation. Carcinogenesis, 20: 1439-1444, 1999.
17. Pommier, Y., Kerrigan, D., Hartman, K. D., and Glazer, R. I. Phosphorylation of mammalian DNA topoisomerase I and activation by protein kinase C. J. Biol. Chem., 265: 9418-9422, 1990.
18. Yu, D., Khan, E., Khaleque, M. A., Lee, J., Laco, G., Kohlhagen, G., Kharbanda, S., Chengheng, Y.-C., Pommier, Y., and Bharti, A. Phosphorylation of DNA topoisomerase I by the c-Ab1 tyrosine kinase confers camptothecin sensitivity. J. Biol. Chem., 51851-51861, 2004.
19. Rossi, F., Labourier, E., Form, T., Divita, G., Derancourt, J., Riou, J. F., Antoine, E., Cathala, G., Brunel, C., and Tazi, J. Specific phosphorylation of SR proteins by mammalian DNA topoisomerase I. Nature Biotechnology, 381: 80-82, 1996.
20. Merino, A., Madden, K. R., Lane, W. S., Champoux, J. J., and Reinberg, D. DNA topoisomerase I is involved in both repression and activation of transcription. Nature Biotechnology, 365: 227-232, 1993.
21. Hsiang, Y. H., Lihou, M. G., and Liu, L. F. Arrest of replication forks by drug-stabilized topoisomerase I-DNA cleavable complexes as a mechanism of cell killing by camptothecin. Cancer Res., 49: 5077-5082, 1989.
22. Strumberg, D., Pilon, A. A., Smith, M., Hickey, R., Malkas, L., and Pommier, Y. Conversion of Topoisomerase I Cleavage Complexes on the Leading Strand of Ribosomal DNA into 5′-Phosphorylated DNA Double-Strand Breaks by Replication Runoff. Mol. Cell. Biol., 20: 3977-3987, 2000.
23. Kang, M. R., Muller, M. T., and Chung, I. K. Telomeric DNA Damage by Topoisomerase I: A possible mechanism of killing by camptothecin. J. Biol. Chem., 279: 12535-12541, 2004.
24. Redinbo, M., Stewart, L., Kuhn, P., Champoux, J., and Hol, W. Crystal structures of human topoisomerase I in covalent and noncovalent complexes with DNA. Science, 279: 1504-1513, 1998.
25. Stewart, L. and Champoux, J. Purification of baculovirus-expressed human DNA topoisomerase I. Meth. Mol. Biol., 94: 223-234, 1999.
26. Holden, J., Rolfson, D., and Lowe, R. DNA topoisomerase I from human placenta. Biochim. Biophys. Acta, 1049: 303-310, 1990.
27. Parmley, S. and Smith, G. Filamentous fusion phage cloning vectors for the study of epitopes and design of vaccines. Adv. Exper. Med. Biol., 251: 215-218, 1989.
28. Vives, E., Brodin, P., and Lebleu, B. A truncated HIV-1 Tat protein basic domain rapidly translocates through the plasma membrane and accumulates in the cell nucleus. J. Biol. Chem., 272: 16010-16017, 1997.
29. Marshall, K. M., Matsumoto, S. S., Holden, J. A., Concepcio´n, G. P., Tasdemir, D., Ireland, C. M., and Barrows, L. R. The anti-neoplastic and novel topoisomerase II-mediated cytotoxicity of neoamphimedine, a marine pyridoacridine. Biochem. Pharm., 66: 447-458, 2003.
30. Mossman, T. Rapid colorimetric assay for cellular growth and survival: Application to proliferation and cytotoxicity assays. J. Immunol. Meth., 65: 55-63, 1983.
31. Blander, G., Kipnis, J., Leal, J. F. M., Yu, C.-E., Schellenberg, G. D., and Oren, M. Physical and functional Interaction between p53 and the Werner's Syndrome Protein. J. Biol. Chem., 274: 29463-29469, 1999.
32. Carty, S. M. and Greenleaf, A. L. Hyperphosphorylated C-terminal Repeat Domain-associating Proteins in the Nuclear Proteome Link Transcription to DNA/Chromatin Modification and RNA Processing. Mol Cell Proteomics, 1: 598-610, 2002.
33. Czubaty, A., Girstun, A., Kowalska-Loth, B., Trzcinska, A. M., Purta, E., Winczura, A., Grajkowski, W., and Staron, K. Proteomic analysis of complexes formed by human topoisomerase I. Biochim Biophys Acta., 1749: 133-41, 2005.
34. Gobert, C., Bracco, L., Rossi, F., Olivier, M., Tazi, J., Lavelle, F., Larsen, A., and Riou, J. Modulation of DNA topoisomerase I activity by p53. Biochemistry, 35: 5778-5786, 1996.
35. Gobert, C., Skladanowski, A., and Larsen, A. K. The interaction between p53 and DNA topoisomerase I is regulated differently in cells with wild-type and mutant p53. Proc. Natl. Acad. Sci. USA, 96: 10355-10360, 1999.
36. Jiang, M., Axea, T., Holgate, R., Rubbi, C. P., Okorokov, L. A., Mee, T., and Milner, J. p53 binds the nuclear matrix in normal cells: binding involves the proline-rich domain of p53 and increases following genotoxic stress. Oncogene, 206: 5449-5458, 2001.
37. De Vries, E. M., Ricke, D. O., De Vries, T. N., Hartmann, A., Blaszyk, H., Liao, D., Soussi, T., Kovach, J. S., and Sommer, S. S. Database of mutations in the p53 and APC tumor suppressor genes designed to facilitate molecular epidemiological analyses. Hum Mutat, 7( ): 202-13, 1996.
38. Rahden-Staron, I., Szumilo, M., Grosicka, E., Kraakman van der Zwet, M., and Zdzienicka, M. Z. Defective BRCA2 influences topoisomerase I activity in mammalian cells. Acta Biochim. Pol., 50: 139-144, 2003.

What is claimed is:

1. An isolated peptide comprising an amino acid sequence selected from the group consisting of: SEQ ID NOS: 1-30, an amino acid sequence at least about 90% identical to the amino acid sequence of SEQ ID NOS: 1-30, or the amino acid sequence of SEQ ID NOS: 1-30 having one or more conservative amino acid substitutions.

2. The isolated peptide of claim 1, wherein the peptide interacts with topoisomerase-I.

3. The isolated peptide of claim 1, wherein the peptide enhances topoisomerase-I interacting compositions.

4. The isolated peptide of claim 3, wherein the topoisomerase-I interacting composition comprises camptothecin.

5. A vector comprising the isolated peptide of claim 1.

6. A cell comprising the vector of claim 5.

7. A composition comprising the peptide of claim 1.

8. The composition of claim 7, further comprising another cancer-treating composition

9. A method of treating a disease associated with topoisomerase I, the method comprising:
identifying a subject having a disease associated with the topoisomerase I; and
administering to the subject a composition comprising the peptide of claim 1.

10. The method of claim 9, wherein the subject has cancer.

11. The method of claim 9, wherein the peptide is administered simultaneously with another topoisomerase-I interacting composition.

12. The method of claim 10, wherein the topoisomerase-I interacting composition comprises camptothecin.
